(19) United States
(12) Patent Application Publication
(10) Pub. No.: US 2011/0105798 A1
(43) Pub. Date: May 5, 2011

(54) SYNTHESIS OF CHIRAL AMINES

(76) Inventors: Paul O'Shea, Quebec (CA); Francis Gosselin, Quebec (CA); James C. Mcwilliams, Upper Saddle River, NJ (US)

(21) Appl. No.: 13/000,372

(22) PCT Filed: Jun. 22, 2009
(86) PCT No.: PCT/US09/48129

§ 371 Date: Dec. 21, 2010

Related U.S. Application Data
(60) Provisional application No. 61/133,287, filed on Jun. 27, 2008.

(51) Int. Cl. C07C 211/27

(52) U.S. Cl. 564/374

(57) ABSTRACT

The instant invention involves the enantioselective hydrogenation of isomeric N—H imines (N-unsubstituted) using a transition metal based catalyst modified with a chiral phosphine derivative to produce enantiomerically enriched chiral amines.

### BACKGROUND OF THE INVENTION

The instant invention involves the enantioselective hydrogenation of isomeric N—H imines (N-unsubstituted) using a transition metal based catalyst modified with a chiral phosphine derivative to produce enantiomerically enriched chiral amines.

The enantioselective reduction of imines poses a considerable synthetic challenge and is currently the subject of research efforts worldwide. Currently known procedures involve additional steps for the installation of a protecting group and subsequent removal after reduction. The instant invention provides a means to prepare N—H ketoimines as stable hydrochloride salts and reduction without the need for protection and deprotection steps.

### SUMMARY OF THE INVENTION

By this invention, there are provided processes for the preparation of compounds of formula I:

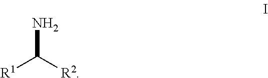

comprising the steps of:

a. Mixing an NH-imine of formula II with an organic solvent and a chiral transition metal catalyst, and

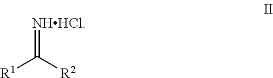

b. Reducing the NH-imine of formula II via pressurization with H₂ to produce the compound of formula I;
wherein R¹ is C₁₋₆ alkyl, C₁₋₆ haloalkyl or aryl, wherein said aryl is optionally substituted with one to three substituents independently selected from the group consisting of halo, C₁₋₃ alkyl, C₁₋₅ haloalkyl, —O(C₁₋₃ alkyl) and —SOₘ(C₁₋₃ alkyl);
R² is C₁₋₆ alkyl;
m is an integer from zero to two.

### DETAILED DESCRIPTION OF THE INVENTION

By this invention, there are provided processes for the preparation of compounds of formula I:

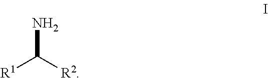

comprising the steps of:

a. Mixing an NH-imine of formula II with an organic solvent and a chiral transition metal catalyst, and

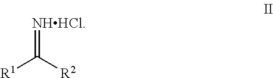

b. Reducing the NH-imine of formula II via pressurization with H₂ to produce the compound of formula I;
wherein R¹ is C₁₋₆ alkyl, C₁₋₆ haloalkyl or aryl, wherein said aryl is optionally substituted with one to three substituents independently selected from the group consisting of halo, C₁₋₃ alkyl, C₁₋₅ haloalkyl, —O(C₁₋₃ alkyl) and —SOₘ(C₁₋₃ alkyl); R² is C₁₋₆ alkyl;
m is an integer from zero to two.

In an embodiment of the invention, the organic solvent is selected from the group consisting of 1,2-diehloroethane, dichloromethane, chlorobenzene, 2,2,2-trifluoroethanol, hexafluoroisopropanol, acetic acid, methanol, ethanol, 2-propanol, tetrahydrofuran, 2-methyltetrahydrofuran, teat-butyl methyl ether (MTBE) and mixtures thereof. In a class of the invention, the organic solvent is 1,2-dichloroethane or 2,2,2-trifluoroethanol.

In an embodiment of the invention, the chiral transition metal catalyst includes, but is not limited to ruthenium catalysts, iridium catalysts, rhodium catalysts, palladium catalysts and mixtures thereof. For example, [Ir(cod)₂Cl]₂ and Ir(cod)₂BF₄ can be combined as appropriate with a suitable chiral phosphine derivative, or alternatively one can use pre-formed chiral catalysts such as (R)-[(Me-BPE)Rh(cod)BF₄] or [(R)-(tol-BINAP)RuCl₂]₂.Et₃N. In a class of the invention, the chiral transition metal catalyst includes, but is not limited to (R)-[(Me-BPE)Rh(cod)BF₄], [Ir(cod)₂Cl]₂combined with (R,S)—PFP—P(tBu)₂, [(R)-(tol-BINAP)RuCl₂]₂.Et₃N, and Ir(cod)₂BF₄ combined with (R,S)—PFP—P(tBu)₂.

In an embodiment of the invention, the pressurization with H₂ is performed between 150 and 500 psi.

In an embodiment of the invention, the pressurization with H₂ is performed between 0° C. to 150° C. In a class of the invention, the pressurization with H₂ is performed between 25° C. to 40° C. In a subclass of the invention, the pressurization with H₂ is performed at 40° C.

The term “alkyl” as used herein shall mean a substituting univalent group derived by conceptual removal of one hydrogen atom from a straight or branched-chain acyclic saturated hydrocarbon (i.e., —CH₃, —CH₂CH₃, —CH₂CH₂CH₃, —CH(CH₃)₂, —CH₂CH₂CH₂CH₃, —CH₂CH(CH₃)₂, —C(CH₃)₃, etc).

As used herein, “aryl” is intended to mean any stable monocyclic or bicyclic carbon ring of up to 12 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

As appreciated by those of skill in the art, “halo” or “halogen” as used herein is intended to include chloro, fluoro, bromo and iodo. The term “keto” means carbonyl (C═O). The term “alkoxy” as used herein means an alkyl portion, where alkyl is as defined above, connected to the remainder of the molecule via an oxygen atom. Examples of alkoxy include methoxy, ethoxy and the like.

The term “haloalkyl” means an alkyl radical as defined above, unless otherwise specified, that is substituted with one to five, preferably one to three halogen. Representative examples include, but are not limited to trifluoromethyl, dichloroethyl, and the like.

In the schemes and examples below, various reagent symbols and abbreviations have the following meanings:

DCE: 1,2-dichloroethane

TFE: 2,2,2-trifluoroethanol

MeOH: methanol

cod: cyclooctadiene

(R)—(S)—PFP—P(tBu)₂: (R)-1-[(S)-diphenylphosphinoferrocenyl]ethyldi-tert-butyl-phosphine

BF₄: tetrafluoroborate

(R)-MeBPE: 1,2-bis[(R,R)-trans-2,5-dimethyl-1-phospholanol]ethane

(R)-TolBINAP: (R)-(+)-2,2′-bis(di-para-tolylphosphino)-1-1′-binaphthyl

The compounds of the present invention can be prepared according to the following general scheme, using appropriate materials, and are further exemplified by the subsequent specific examples. The compounds illustrated in the examples are not, however, to be construed as forming the only genus that is considered as the invention. Those skilled in the art will readily understand that known variations of the conditions and processes of the following preparative procedures can be used to prepare these compounds. All temperatures are degrees Celsius unless otherwise noted.

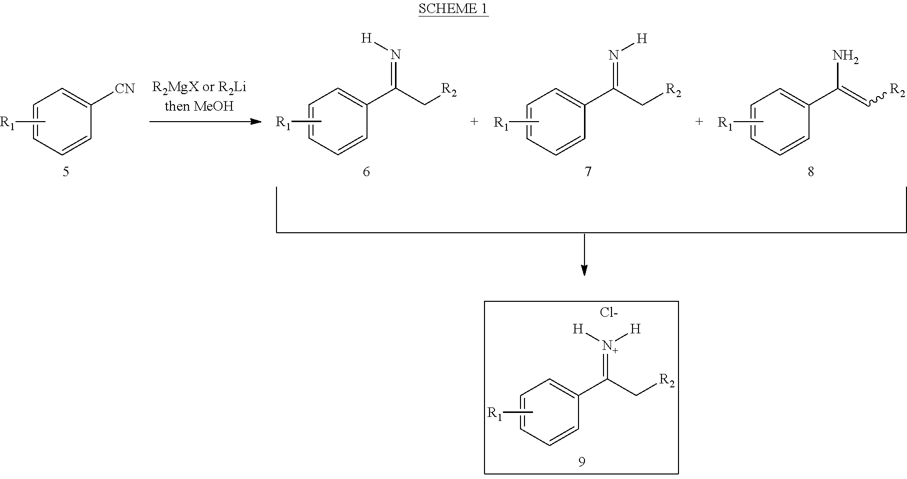

Scheme 1 describes the preparation of NH imines. The NH imines are prepared by addition of a suitable organometallic reagent to nitriles. Quenching of the metallated imine intermediate with methanol and removal of metal salts by filtration affords isomeric NH imine as free bases. Salt formation with anhydrous hydrochloric acid in diethyl ether (Et₂O) of tent-butyl methyl ether (MTBE) affords NH imines hydrochloride salts as free-flowing white solids.

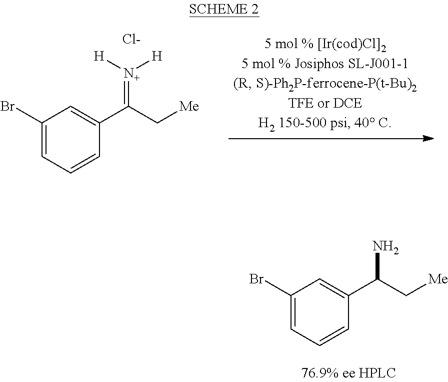

Scheme 2 describes the enantioselective hydrogenation of NH imines. The hydrogenation is performed under inert atmosphere by mixing the transition metal pre-catalyst and chiral phosphine ligand in a suitable solvent, adding the NH imine hydrochloride salt and pressurizing the vessel with H₂ gas. After the specified reaction time the reactor is vented and the reaction mixture is analyzed by HPLC.

### EXAMPLE 1

### Preparation of 1-(3-Bromophenyl)-1-Propylamine

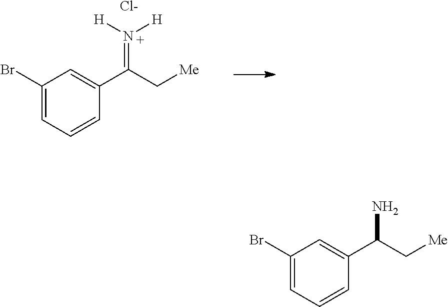

In a vial equipped with a stir bar was charged anhydrous 1,2-DCE or TFE (1 mL), [Ir(cod)₂Cl]₂ (5 mol %), (R,S)—PFP—P(tBu)₂ (SL-J002-1, 5 mol %) and substrate NH-imine hydrochloride salt (0.1 mmol). The mixture was stirred for 5 min and then pressurized with H₂ at 150-500 psi and 25-40° C. After stirring 20 h, the H₂ pressure was relieved and the mixture was analyzed by reverse-phase HPLC (71% conversion) and chiral HPLC (76.9% ee).

### EXAMPLE 2

### Preparation of 1-(3-Bromophenyl)-1-Propylamine

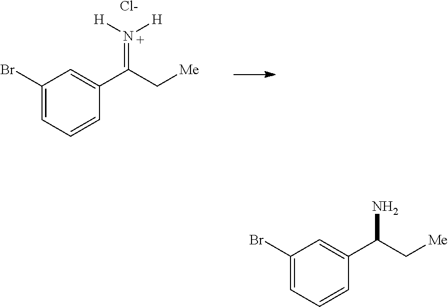

In a vial equipped with a stir bar was charged anhydrous MeOH (1 mL), (R)-Me-BPE)Rh(cod)BF₄ (5 mol %) and substrate NH-imine hydrochloride salt (0.1 mmol). The mixture was stirred for 5 min and then pressurized with H₂ at 150-500 psi and 25-40° C. After stirring 20 h, the H₂ pressure was relieved and the mixture was analyzed by reverse-phase HPLC (100% conversion) and chiral HPLC (43.1% ee).

### EXAMPLE 3

### Preparation of 1-(3-Bromophenyl)-1-Propylamine

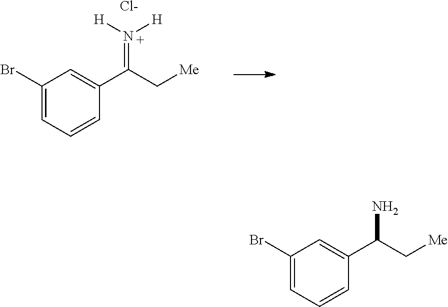

In a vial equipped with a stir bar was charged anhydrous trifluoroethanol (1 mL), [(R)-(tol-BINAP)RuCl₂]₂.Et₃N (5 mol %) and substrate NH-imine hydrochloride salt (0.1 mmol). The mixture was stirred for 5 min and then pressurized with H₂ at 150-500 psi and 25-40° C. After stirring 20 h, the H₂ pressure was relieved and the mixture was analyzed by reverse-phase HPLC (76% conversion) and chiral HPLC (38.6% ee).

### EXAMPLE 4

### Preparation of 1-(3-Bromophenyl)-1-Propylamine

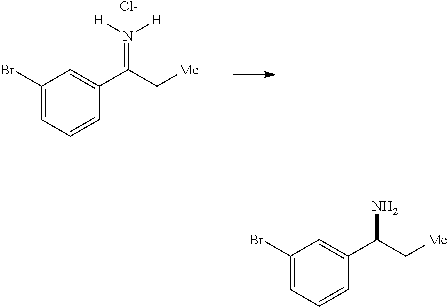

In a vial equipped with a stir bar was charged anhydrous 1,2-DCE (1 mL), Ir(cod)₂BF₄ (5 mol %), (R,S)—PFP—P(tBu)₂ (SL-J002-1, 5 mol %) and substrate NH-imine hydrochloride salt (0.1 mmol). The mixture was stirred for 5 min and then pressurized with H₂ at 150-500 psi and 25-40° C. After stirring 20 h, the H₂ pressure was relieved and the mixture was analyzed by reverse-phase HPLC (59% conversion) by chiral HPLC (29.8% ee).

What is claimed is:

1. A processes for the preparation of a compound of formula I: 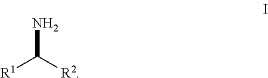
comprising the steps of:
a. Mixing an NH-imine of formula II with an organic solvent and a chiral transition metal catalyst, and 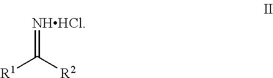
b. Reducing the NH-imine of formula II via pressurization with H₂ to produce the compound of formula I;
wherein R¹ is C₁₋₆ alkyl, C₁₋₆ haloalkyl or aryl, wherein said aryl is optionally substituted with one to three substituents independently selected from the group consisting of halo, C₁₋₃ alkyl, C₁₋₅ haloalkyl, —O(C₁₋₃ alkyl) and —SOₘ(C₁₋₃ alkyl); R² is C₁₋₆ alkyl;
m is an integer from zero to two.

2. The process of claim 1 wherein the organic solvent is selected from the group consisting of 1,2-dichloroethane, dichloromethane, chlorobenzene, 2,2,2-trifluoroethanol, hexafluoroisopropanol, acetic acid, methanol, ethanol, 2-propanol, tetrahydrofuran, 2-methyltetrahydrofuran, tent-butyl methyl ether and mixtures thereof.

3. The process of claim 2 wherein the organic solvent is 1,2-dichloroethane or 2,2,2-trifluorethanol.

4. The process of claim 3 wherein the pressurization with H₂ is performed between 150 and 500 psi.

5. The process of claim 4 wherein the pressurization with H₂ is performed between 0° C. to 150° C.

6. The process of claim 5 wherein the pressurization with H₂ is performed at 40° C.

7. The process of claim 1 wherein the chiral transition metal catalyst is selected from the group consisting of ruthenium catalysts, iridium catalysts, rhodium catalysts, palladium catalysts and mixtures thereof.

8. The process of claim 7 wherein the chiral transition metal catalyst is selected from the group consisting of (R)-[(Me-BPE)Rh(cod)BF₄], [Ir(cod)₂Cl]₂ combined with (R,S)—PFP—P(tBu)₂, [(R)-(tol-BINAP)RuCl₂]₂.Et₃N, and Ir(cod)₂BF₄ combined with (R,S)—PFP—P(tBu)₂.
